(19) United States
(12) Patent Application Publication
(10) Pub. No.: US 2018/0160982 A1
(43) Pub. Date: Jun. 14, 2018

(54) SENSOR FUSION FOR BRAIN MEASUREMENT

(71) Applicant: X Development LLC, Mountain View, CA (US)

(72) Inventors: Sarah Laszlo, Binghamton, NY (US); Philip Edwin Watson, Santa Cruz, CA (US); Matthew Eisaman, Port Jefferson, NY (US); Brian Adolf, San Mateo, CA (US); Gabriella Levine, San Francisco, CA (US)

(21) Appl. No.: 15/374,428

(22) Filed: Dec. 9, 2016

(51) Int. Cl. A61B 5/00; A61B 5/0478; A61B 5/0205; A61B 5/11

(52) U.S. Cl. CPC A61B 5/7264; A61B 5/0478; A61B 5/0205; A61B 5/021; A61B 5/7225; A61B 5/7278; A61B 5/11

(57) ABSTRACT

Methods, systems, and apparatus, including computer programs encoded on a computer storage medium, for receiving brain activity data of a user from a brainwave sensor and user physiological data from a non-brainwave sensor, where the brain activity data represents a brainwave pattern related to a physiological activity of the user and a brainwave pattern related to a mental activity of the user. Identifying a physiological action of the user based on the user physiological data. Identifying, within the brain activity data, a pattern that is representative of the identified physiological action. Filtering the brain activity data to lessen a contribution of the pattern representative of the identified physiological action to the brain activity data, thereby, providing filtered brain activity data.

### TECHNICAL FIELD

This disclosure generally relates to brainwave measurements. More particularly the disclosure relates to processes for filtering brainwave signals.

### BACKGROUND

Brain activity can be measured using brainwave measurement systems such as electroencephalogram (EEG) machines to measure electrical signals within the brain. The quality of brainwave signal data obtained by EEG systems varies widely. For example, precision laboratory grade EEG systems tend to produce clean, high-quality data, while non-laboratory grade systems produce noisy data streams.

### SUMMARY

This specification describes systems, methods, devices, and other techniques for using sensor fusion to filter brainwave data. More specifically, implementations use data from non-brainwave sensors to identify a user activity that adds noise to brainwave data received from a brainwave sensor (e.g., EEG electrode(s) or an EEG system). An exemplary system uses the non-brainwave sensor data to identify signal patterns in the brainwave data that correlate to the identified user activity and filters the brainwave data to reduce the effects of the signal patterns associated with the user activity on the brainwave data. For example, the effects of signal patterns related to muscular movements on brainwave data can be reduced or removed to yield signals that are more representative of cephalic brainwaves.

In some implementations, a system employs a machine learning algorithm to fuse data from one or more sensors with brainwave data. The system can correlate the non- brainwave data with related brainwave data. Once correlated, the system can filter the brainwave data stream appropriately. For example, in a given situation desired brainwave data may include data related to a user's alertness or non-muscular mental functions (e.g., Alpha waves). However, Alpha wave data may be heavily masked by noise due to user movement and interfering signals related to undesired brain functions, e.g., controlling head movements, facial movements, eye movements, and heart-beat. The system can use data from non-brain sensors (e.g., cameras, accelerometers, etc.) to detect such occurrences based on external physical actions of the user. For example, when video or accelerometer data indicates that a user moved their head, the system and can correlate the timing of such data to the timing of the brainwave data. The system can then identify the undesired brain activity in the brainwave data stream and filter the undesired data. For example, the system can apply appropriate filters to the brainwave data to remove brain waves that are associated with the head motion while retaining the Alpha waves. Such filters may be initialized based on known brain wave patterns for muscle control (e.g. head motion) and further refined based on learned analysis of a particular user's brain wave patterns. The above processes can be performed on data from each of multiple brainwave sensors individually.

In some implementations, the system can be integrated into a wearable device that is communicatively linked to a personal computing devices (e.g., through a wired or wireless communication link). A wearable device can incorporate comb-like brainwave sensors that measure brain waves through contact with a user's scalp. Some implementations can include retractable (non-puncture) needle electrodes that contact the user's scalp. The wearable device can include non-brainwave sensors such as accelerometers to monitor the user's head movements. Additional, non- brainwave sensors can include a camera on the personal computing device to detect facial movements and eye motion to filter related brain waves from the brainwave data. Implementations, can detect heart-beat by directly measuring a user's pulse or by characteristics of heartbeat from images of the user (e.g., slight changes in completion or pulsations in blood vessels).

In general, innovative aspects of the subject matter described in this specification can be embodied in methods that include the actions of receiving brain activity data of a user from a brainwave sensor and user physiological data from a non-brainwave sensor, where the brain activity data represents a brainwave pattern related to a physiological activity of the user and a brainwave pattern related to a mental activity of the user. Identifying a physiological action of the user based on the user physiological data. Identifying, within the brain activity data, a pattern that is representative of the identified physiological action. Filtering the brain activity data to lessen a contribution of the pattern representative of the identified physiological action to the brain activity data, thereby, providing filtered brain activity data. Other implementations of this aspect include corresponding systems, apparatus, and computer programs, configured to perform the actions of the methods, encoded on computer storage devices. These and other implementations can each optionally include one or more of the following features.

In some implementations, the non-brainwave sensor includes a sensor such as a motion sensor, an accelerometer, a camera, a radar sensor, a microphone, a blood pressure sensor, a pulse sensor, and a skin conductance sensor.

In some implementations, physiological action includes an action such as a head movement, a movement of facial muscles, a pulse rate, and an eye movement.

In some implementations, identifying the pattern that is representative of the identified physiological action and filtering the brain activity data are performed by a machine learning system. In some implementations, the machine learning system is a feed forward auto encoder neural network.

Some implementations include identifying a brain state of the user based on correlation between the identified physical action and the filtered brain activity data.

In some implementations, the identified physiological action is an action such as eye movement, a blink rate, perspiration, and a keyboard typing intensity, and wherein the brain state is a level of user attentiveness.

Some implementations include prompting the user to perform an action based on determining the brain state of the user.

In some implementations, the brainwave sensor is part of a brainwave sensor system and the brain activity data is received from the brainwave sensor system.

In some implementations, the brainwave sensor system is a wearable brainwave sensor system that includes a plurality of electrodes arranged in a comb-like structure. In some implementations, the electrodes are retractable. In some implementations, the non-brainwave sensor is a motion sensor mounted on the brainwave sensor system.

Another general of the subject matter described in this specification can be embodied in a system that includes a brainwave sensor, at least one non-brainwave sensor, and a data processing module. The data processing module is communicably coupled to the brainwave sensor and the at least one non-brainwave sensor. The data processing module includes a physiological action detection module and a filtering module. The physiological action detection module is configured to identify a physiological action of the user based on user physiological data received from the at least one non-brainwave sensor. The filtering module is configured to identify a pattern representative of the physiological action of the user, within brain activity data received from the brainwave sensor, and filter the brain activity data to lessen a contribution of the pattern representative of the identified physiological action to the brain activity data to provide filtered brain activity data. This and other implementations can each optionally include one or more of the following features.

In some implementations, the data processing module includes a data fusion module that is configured to identify a brain state of the user based on a correlation between the physiological data and the filtered brain activity data.

In some implementations, the data processing module includes an output module that is configured to present, to a user, a prompt to perform an action based on the determined brain state of the user.

In some implementations, the non-brainwave sensors include a sensor such as a motion sensor, an accelerometer, a camera, a radar sensor, a microphone, a blood pressure sensor, a pulse sensor, and a skin conductance sensor.

In some implementations, the physiological action includes an action such as a head movement, a movement of facial muscles, a pulse rate, and an eye movement.

In some implementations, the filtering module comprises a machine learning system. In some implementations, the machine learning system is configured to identify the pattern that is representative of the identified physiological action and filter the brain activity data.

Particular implementations of the subject matter described in this specification can be implemented so as to realize one or more of the following advantages. Implementations of the present disclosure improve the signal quality of brainwave sensors and brainwave sensor systems. Implementations may permit the acquisition of high quality brainwave data while a user is ambulatory. Implementations may enable transparent co-registration of eye movements with EEG activity.

The details of one or more implementations of the subject matter of this disclosure are set forth in the accompanying drawings and the description below. Other features, aspects, and advantages of the subject matter will become apparent from the description, the drawings, and the claims.

### DESCRIPTION OF DRAWINGS

FIG. 1 depicts block diagram of an example system for filtering brainwave data in accordance with implementations of the present disclosure.

FIG. 2 depicts an example brainwave sensor system according to implementations of the present disclosure.

FIGS. 3A and 3B depict example brainwave data signals according to implementations of the present disclosure.

FIG. 4 depicts a flowchart of an example process for filtering brainwave data in accordance with implementations of the present disclosure.

FIG. 5 depicts a schematic diagram of a computer system that may be applied to any of the computer-implemented methods and other techniques described herein.

Like reference numbers and designations in the various drawings indicate like elements.

### DETAILED DESCRIPTION

As used herein the term “filtering” as applied to brainwave data is not limited to filtering in the spectral domain such filtering a signal based on frequency components. The term filtering includes removing or reducing the effects of undesired signals from a brainwave data signal. For example, as described in more detail below, filtering brainwave signals includes removing or reducing the effects of signals or noise present in the brainwave data due to other physiological actions of a user.

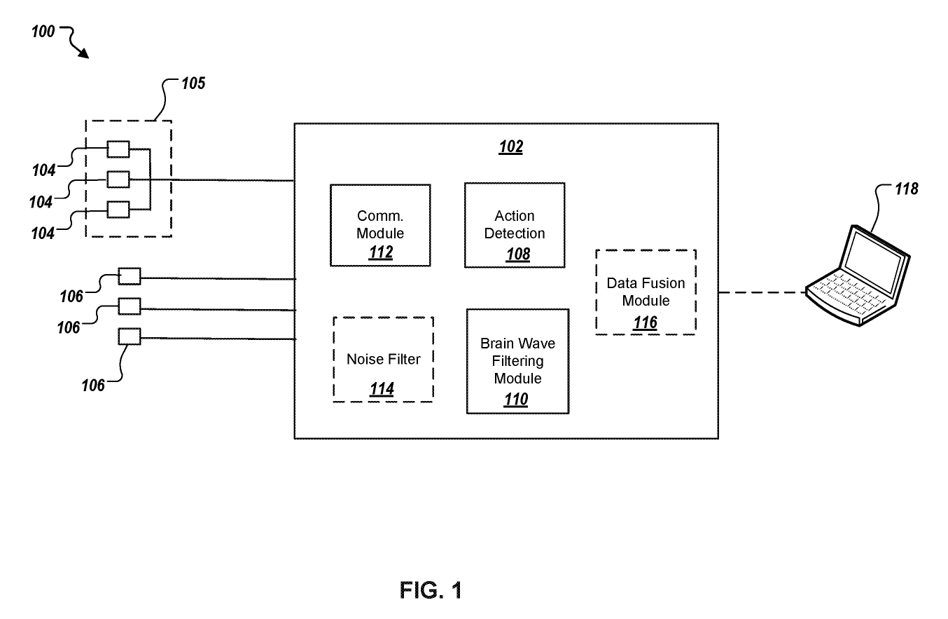

FIG. 1 depicts a block diagram of an example system 100 for filtering brainwave data in accordance with implementations of the present disclosure. The system includes a brainwave data processing module 102 which is in communication with brainwave sensors 104 and non-brainwave sensors 106. The data processing module 102 can be implemented as a hardware or a software module. For example, the data processing module can be a hardware or software module that is incorporated into a computing system such as a brainwave monitoring system, a desktop or laptop computer, or a wearable device. The data processing module 102 includes several sub-modules which are described in more detail below. As a whole, the data processing module 102 receives user brainwave data from the brainwave sensors 104 and data related to other physiological actions of the user from the non-brainwave sensors 106. The data processing module 102 uses the data from the non-brainwave sensors 106 to filter the brainwave data.

For example, user physiological actions such as muscular movements (e.g., in the face, head, and eyes), heartbeats, and respiration can create noise in the brainwave signals received by brainwave sensors 104. The noise may be due to other electrical signals in the body (e.g., nervous system impulses to control muscle movements) that interfere with the brainwave data, other brain signals for controlling such physiological actions, or both.

The data processing module 102 uses the data from non-brainwave sensors 106 to identify different user physiological actions and remove or, at least, reduce the effects that such user actions have on the brainwave data. For example, the data processing module 102 can use data from the non-brainwave sensors 106 to filter noise due to user movements from the brainwave data.

User head movements is one example of user movements that may create noise in the brainwave data. Accordingly, in some embodiments, the data processing module 102 uses data from the non-brainwave sensors 106 to detect a user head movement and remove or reduce the effects of the head movement on the brainwave data.

In some implementations, the data processing module 102 is used to remove undesired brain activity signals from the brainwave data. For example, the brainwave data may capture brainwaves associated with both brain activity and other physiological activity (e.g., muscular activity). The data processing module 102 can use the data from the non-brainwave sensors 106 to identify a user's muscular activity (e.g., limb and facial movements, heartbeat, respiration, eye movements, etc.), identify signal patterns associated with an identified muscular activity, and filter the brainwave data to remove or reduce the effects of such signal patterns on the brainwave data.

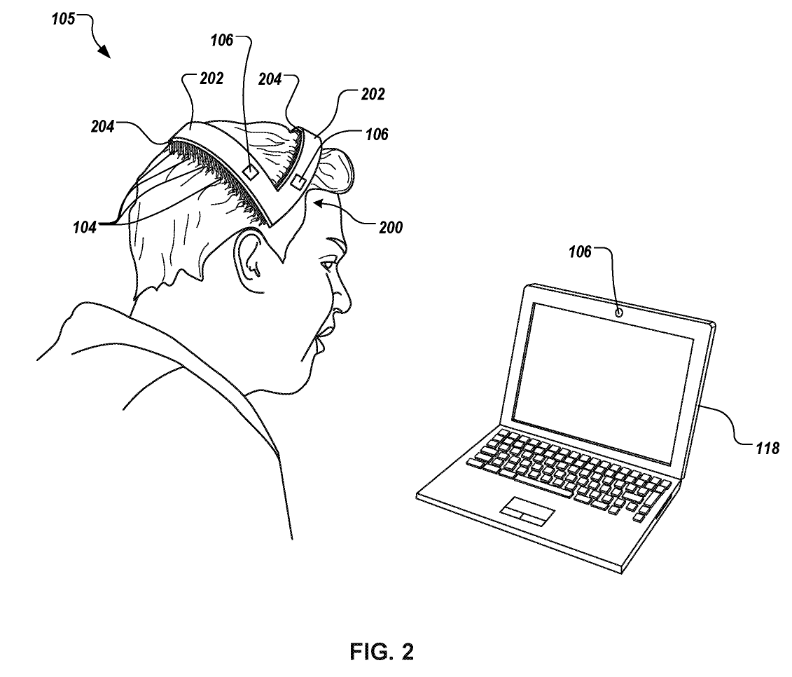

In general, any sensors capable of detecting brainwaves may be used. For example, the brainwave sensors 104 can be one or more individual electrodes (e.g., multiple EEG electrodes) that are connected to the data processing module 102 by wired connection. The brainwave sensors 104 can be part of a brainwave sensor system 105 that is in communication with the data processing module 102. A brainwave sensor system 105 can include multiple individual brainwave sensors 104 and computer hardware (e.g., processors and memory) to receive, process, and/or display data received from the brainwave sensors 104. Example brainwave sensor systems 105 can include, but are not limited to, EEG systems, a wearable brainwave detection device (e.g., as described below in reference to FIG. 2 below), a magnetoencephalography (MEG) system, and an Event-Related Optical Signal (EROS) system, sometimes also referred to as “Fast NIRS” (Near Infrared spectroscopy). A brainwave sensor system 105 can transmit brainwave data to the data processing module 102 through a wired or wireless connection.

FIG. 2 depicts an example brainwave sensor system 105. The sensor system 105 is a wearable device 200 which includes a pair of bands 202 that fit over a user's head. Specifically, the wearable device 200 includes one band which fits over the front of a user's head and the other band 202 which fits over the back of a user's head, securing the device 200 sufficiently to the user during operation. The bands 202 include a plurality of brainwave sensors 104. The sensors 104 can be, for example, electrodes configured to sense the user's brainwaves through the skin. For example, the electrodes can be non-invasive and configured to contact the user's scalp and sense the user's brainwaves through the scalp. In some implementations, the electrodes can be secured to the user's scalp by an adhesive.

The sensors 104 are distributed across the rear side 204 of each band 202. In some examples, the sensors 104 can be distributed across the bands 202 in to form a comb-like structure. For example, the sensors 104 can be narrow pins distributed across the bands 202 such that a user can slide the bands 202 over their head allowing the sensors 104 to slide through the user's hair, like a comb, and contact the user's scalp. Furthermore, the comb-like structure sensors 104 distributed on the bands 202 may enable the device 200 to be retained in place on the user's head by the user's hair. In some implementations, the sensors 104 are retractable. For example, the sensors 104 can be retracted into the body of the bands 202.

The wearable device 200 is in communication with a computing device 118, e.g., a laptop, tablet computer, desktop computer, smartphone, or brainwave data processing system. For example, the data processing module 102 can be implemented as a software application on a computing device 118. The wearable device 200 communicates brainwave data received from the sensors 104 to the computing device 118. In some implementations, the data processing module 102 can be implemented as a hardware or software module on the wearable device 200. In such implementations, the device 200 can communicate filtered brainwave data to the computing device 118 for use by other applications on the computing device, e.g., medical applications, brainwave monitoring applications, research applications.

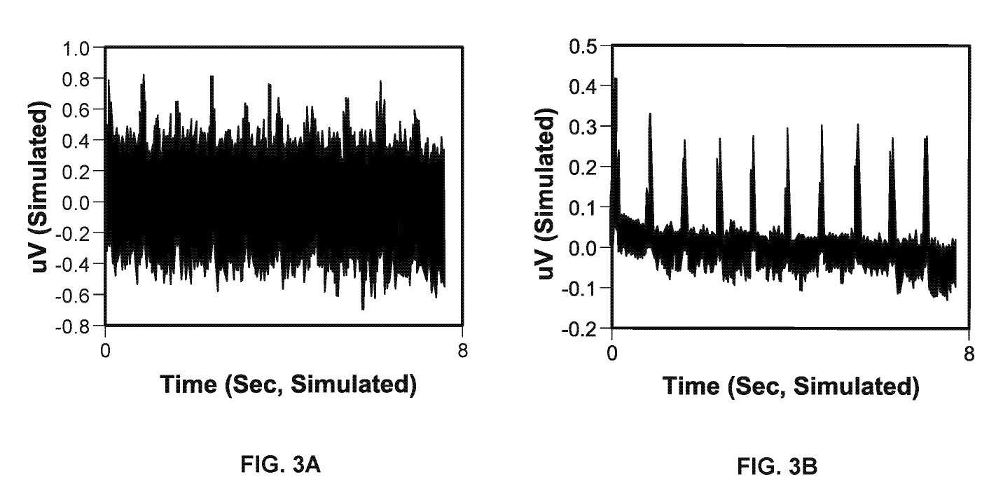

FIG. 3A illustrates a simulated example of noisy brainwave data that may be received from one brainwave sensor. The signal in FIG. 3A represents an aggregate electrical signal that can include multiple signal patterns related to both physiological activities of the user and brainwave patterns related to mental activities of the user. Each of the signal patterns may not be easily recognizable. Furthermore, the signal patterns may interfere with each other. For example, a signal pattern related to the physiological activity of the user may be viewed as noise with respect to a signal pattern related to the mental activity of the user if the later is desired for further analysis in a given context. On the contrary, the signal pattern related to the mental activity of the user may be viewed as noise with respect to the signal pattern related to the physiological activity of the user if it is the desired signal pattern for further analysis in a different context.

The brainwave sensors 104 or sensor system 105 transmit signals such as the example data signal shown in FIG. 3A to the data processing module 102. The data processing module 102 uses data from other non-brainwave sensors 106 to remove noise and other undesired signal patterns, e.g., signal patterns due to the user's physiological actions, from the brainwave data to produce filtered brainwave data such as that shown in FIG. 3B. FIG. 3B illustrates a simulated example of filtered brainwave data after processing by a data processing module 102.

Referring to FIGS. 1 and 2, the non-brainwave sensors 106 can include, but are not limited to, a motion sensor, an accelerometer, a camera, an infrared camera, a radar sensor, a microphone, a blood pressure sensor, a pulse sensor, a skin conductance sensor, or combination thereof. The non-brainwave sensors 106 can be separate individual sensors, e.g., a webcam on a laptop and an accelerometer in a wearable device 200. The non-brainwave sensors 106 can be combined in one or more devices, e.g., accelerometer(s) mounted on a brainwave sensor system 105 such as a wearable brainwave sensor system 200 to detect head movements, a webcam and/or microphone of a user's computing device 118. For example, FIG. 2 illustrates non-brainwave sensors 106 (e.g., accelerometers) mounted on the band 202 of the wearable device 200. The wearable device 200, in such implementations, may also communicate the non-brainwave data obtained by the non-brainwave sensors 106 to the computing device 118, e.g., if the data processing module 102 is implemented on the computing device 118.

Referring to FIG. 1, the data processing module 102 includes several sub-modules, each of which can be implemented in hardware or software. The data processing module 102 includes an action detection module 108, a brainwave filtering module 110, a communication module 112, and optionally includes a noise filter 114 and/or a data fusion module 116.

The action detection module 108 identifies user physiological actions based on data from one or more of the non-brainwave sensors 106. For example, the action detection module 108 analyzes data from the non-brainwave sensors 106 to identify user physiological actions that may add noise or other undesirable signal patterns to the brainwave data. Examples of such physiological actions can include, but are not limited to, head movements, movements of facial muscles, a pulse rate (e.g., heartbeat), eye movements, respiration, or a combination thereof. For example, the action detection module 108 can identify that a particular type user physiological action has occurred and pass relevant information related to the action to the brainwave filtering module 110.

The action detection module 108 can use image data (e.g., video frames) from a camera using image processing algorithms to identify actions such as head movements, changes in expression that indicate facial muscle movements, and movements of limbs. For example, the action detection module 108 can employ a facial detection algorithm to identify head and limb movements and changes in expression. The action detection module 108 can employ an eye tracking algorithm to identify user eye movements. In some implementations, the action detection module 108 can use a pulse detection algorithm to identify a user's pulse and heart beat based on changes in skin completion. For example, a pulse detection algorithm can be employed to pulse and heartbeat by filtering and amplifying slight variations in color due to the blood flow.

As another example, the action detection module 108 can use accelerometer data to identify user movements. For example, the action detection module 108 can identify user head movements based on data from accelerometers attached to wearable devices such as, a wearable brainwave sensor system 105, a watch, a virtual reality headset, a wireless headset (e.g., bone conduction headphones), a wearable personal fitness device.

Upon identifying a user physiological action, the action detection module 108 provides an indication of a user physiological action to the brainwave filtering module 110. The indication of the physiological action can include the type of physiological action and, in some implementations, timing information related to when the action occurred. The action detection module 108 can pass relevant portion of non-brainwave sensor data to the brainwave filtering module 110.

The brainwave filtering module 110 identifies signal patterns within the brainwave data that are representative of the identified physiological action and filters the brainwave data to reduce or remove the effects of the identified signal patterns. For example, the brainwave filtering module 110 can correlate a particular type of user physiological action (e.g., a head movement) to known signal patterns within the brainwave data that are correlate to the particular type of action. For example, the brainwave filtering module 110 can correlate the timing of an identified head movement with changes in the brainwave signal that correlate with the timing of the head movement.

The brainwave filtering module 110 can utilize a library of signal characteristics representative of different types of signal patterns that occur in brainwave data due to particular types of physiological actions. The brainwave filtering module can use an identification algorithm such as a cross correlation process to identify signal patterns within the brainwave data that correlate with the known characteristic of the particular type of physiological action within a confidence threshold. For example, the brainwave filtering module 110 may include signal characteristics of a patterns representative of a heartbeat. The brainwave filtering module 110 can using timing information from the action detection module 108 to estimate the timing of signal pattern representative of a user's heartbeat within the brainwave data. The brainwave filtering module 110 can correlate the known signal characteristics with the actual signal patterns of the user's heartbeat in the brainwave data to identify the actual heartbeat interference signals in the brainwave data.

The brainwave filtering module 110 then reduces or removes the effects of the identified signal patterns. For example, the brainwave filtering module 110 can reduce the effects of the identified signal patterns by applying machine learning to portions of the brainwave data in which the identified signal patterns occur.

The brainwave filtering module 110 can use various filtering techniques to filter the data. For example, the brainwave filtering module 110 can use matched filters to reduce the effects of an identified signal pattern, canonical artifact waveshapes to remove aspects of the identified signal pattern which correlate with known stereotyped waveshapes, band pass filters to remove spectral effects of the identified signal pattern, or a combination thereof. In some implementations, the brainwave filtering module 110 can subtract the identified signal patterns from the appropriate portions of brainwave data to reduce the effects of an identified signal pattern. For example, a library of signal patterns may be adapted to a particular user over time (e.g., by using a machine learning system or algorithm as discussed in more detail below). Such signal patterns, once identified, can be subtracted from the appropriate portions of the brainwave data (e.g., the portions of the brainwave data in which the signal patterns are identified), or removed by more sophisticated means than subtraction, e.g., independent components analysis.

In some implementations, the brainwave filtering module 110 incorporates a machine learning model to identify signal patterns associated with user physiological activities within the brainwave data and filter the brainwave data to reduce or remove the effects of such signal patterns on the brainwave data. For example, the brainwave filtering module 110 can include a machine learning model that has been trained to receive model inputs, e.g., detection signal data, and to generate a predicted output, e.g., signal patterns associated with particular types of user physiological actions and/or filtered brainwave data in which the effects of such signal patterns are reduced or removed from the brainwave data. In some implementations, the machine learning model is a deep learning model that employs multiple layers of models to generate an output for a received input. The machine learning model may be a deep learning neural network. A deep neural network is a deep machine learning model that includes an output layer and one or more hidden layers that each apply a non-linear transformation to a received input to generate an output. In some cases, the neural network may be a recurrent neural network. A recurrent neural network is a neural network that receives an input sequence and generates an output sequence from the input sequence. In particular, a recurrent neural network uses some or all of the internal state of the network after processing a previous input in the input sequence to generate an output from the current input in the input sequence. In some other implementations, the machine learning model is a shallow machine learning model, e.g., a linear regression model or a generalized linear model.

The machine learning model can be a feed forward auto encoder neural network. For example, the machine learning model can be a three-layer auto encoder neural network. The machine learning model may include an input layer, a hidden layer, and an output layer. In some implementations, the neural network has no recurrent connections between layers. Each layer of the neural network may be fully connected to the next, e.g., there may be no pruning between the layers. The neural network may include an ADAM optimizer for training the network and computing updated layer weights. In some implementations, the neural network may apply a mathematical transformation, e.g., convolutional, to input data prior to feeding the input data to the network.

In some implementations, the machine learning model can be a supervised model. For example, for each input provided to the model during training, the machine learning model can be instructed as to what the correct output should be. The machine learning model can use batch training, e.g., training on a subset of examples before each adjustment, instead of the entire available set of examples. This may improve the efficiency of training the model and may improve the generalizability of the model. The machine learning model may use folded cross-validation. For example, some fraction (the “fold”) of the data available for training can be left out of training and used in a later testing phase to confirm how well the model generalizes.

For example, a machine learning model can be trained to recognize signal patterns associated with various different user physiological actions. For example, the machine learning model can correlate identified user physiological actions with signal patterns within the brainwave data that are related to the identified actions. For example, the machine learning model can be trained to identify noise patterns generated in brainwave sensors when a user moves their head. The machine learning model can be trained to identify interference signal patterns that occur in brainwave that are caused by non-brainwave electrical impulses (e.g., other nervous system signal) in the user's body when the user makes muscular movements (e.g., changing facial expressions, moving their eyes, head or other limbs).

The machine learning model can incorporate the data from the non-brainwave sensors to correlate the timing and/or type of user physiological action with the noise and/or interfere signal patterns associated with such action within the brainwave data. For example, the machine learning model can use non-brainwave data indicating the timing of a user's pulse to identify the start and stop of the user's heartbeat, and correlate known heartbeat signals to signal patterns within the user's brainwave data. The machine learning model can then filter such heartbeat signal patterns from the brainwave data.

As another example, the machine learning model can user non-brainwave data indicating the timing of user head movement to identify the start and stop increased signal noise occurring in the brainwave data due to movements of the brainwave sensors when the user moves their head. The machine learning model can then filter the increased noise from the brainwave data.

In some implementations, the machine learning model can refine the ability to identify signal patterns associated with physiological actions of a particular user. For example, the machine learning model can continue to be trained on user specific data in order to adapt the signal pattern recognition algorithms to the those associated with a particular user. For example, the machine learning model can use brainwave data from periods of time during which the user does not perform any, or performs only few physiological actions. For example, during periods of time when the user is substantially motionless. The machine learning model can use such data to develop a baseline for the user's brainwave data absent noise and interference signal from other (non-brain related) physiological activity. The machine learning model can compare such baseline brainwave data to brainwave data with noise/interference signals due to one or more other physiological actions of the user to more accurately identify the effects of the various different types of user physiological actions on the brainwave data.

The communication module 112 provides a communication interface for the data processing module 102 with the brainwave sensors 104 and/or the non-brainwave sensors 106. The communication module 112 can be a wired communication (e.g., USB, Ethernet) or wireless communication module (e.g., Bluetooth, ZigBee, WiFi). The communication module 112 can serve as an interface with other computing devices 118, e.g., computing devices that may be used to further process or use the filtered brainwave signals. The communication module 112 can be used to communicate directly or indirectly, e.g., through a network, with other remote computing devices 118 such as, e.g., a laptop, a tablet computer, a smartphone, etc.

In some implementations, the data processing module 102 includes a noise filter 114. The noise filter 114 can serve as a pre-filter to remove electromagnetic noise from the brainwave data before it is filtered by the brainwave filtering module 110.

In some implementations, the data processing module 102 includes a data fusion module 110. The data fusion module 116 fuses filtered brainwave data with the non-brainwave sensor data. The data fusion module 116 can be used to identify brain states of a user based on both the filtered brainwave data and data from the non-brainwave sensors 106. For example, the data fusion module 116 can use both the filtered brainwave data and the non-brainwave sensor data to identify user brain states including, but not limited to, attentiveness, tiredness, depth of thought, physiological arousal (e.g., fear or other strong emotions), seizure or pre-seizure activity, or stage of sleep. For example, the data fusion module 116 can use a machine learning model to correlate patterns in the filtered brainwave data and data from the non-brainwave sensors to determine a user's brain state. User physiological actions that may be correlated with brainwave data to determine a user's brain state can include, but are not limited to, head movements, heart rate, eye movement, a blink rate, perspiration, a keyboard typing intensity, or a combination thereof.

For example, a particular pattern of Alpha waves received in conjunction with eye movements focused on a computer screen may indicate that the user has a high level of attentiveness. As another example, a particular pattern of Delta waves received in conjunction with frequent blinking may indicate that the user is tired. For example, a particular pattern of Beta waves received in conjunction with microphone data indicating that a high intensity of keystrokes on a keyboard may indicate that the users is highly focused on a particular task. As another example, a pattern of Alpha waves received in conjunction with quiet accelerometer readings may indicate that the user is asleep. As another example, a pattern of Delta and Sigma waves received just prior to onset of high frequency eye movements may indicate that the user has entered REM sleep. Meanwhile, a burst of Delta and Sigma followed by quiet eye movement readings may indicate that the user has left REM sleep.

In some implementations, the data fusion module 116 can determine an action for a user to take based on determining the user's brain state. For example, if the brainwave and non-brainwave data indicate that the user's level of attentiveness is decreasing, the data fusion module can cause a computing device to prompt the user to take a break. For example, the data fusion module 116 can cause a notification to be displayed on a screen of the user's computing device recommending that the user take a break from working on a computer because the user's attentiveness is decreasing.

In some implementations, the data fusion module 116 can be used to identify non-brainwave sensor data that can serve as proxies for brainwave data. For example, as described above, a burst of Delta and Sigma brain activity followed by detection of rapid eye movements may be indicative of the entrance to REM sleep. The data fusion module 116 may identify that a particular pattern of eye movements is just as predictive of the entrance to REM sleep as the Delta/Sigma burst in combination with eye movements. That is, the eye movements alone may be proxies for the combined brain and eye movement system. Similarly, during REM sleep, the rest of the body (besides the eyes) typically becomes very still. The data fusion module 116 may identify that motion sensing (e.g., by accelerometer data) is also an identification of the start of REM sleep. Thus, for example, the accelerometer data could serve as a proxy for the full brain/eye/muscle system of data.

In an example implementation, the brainwave filtering system 100 can be integrated into a vehicle and used to monitor a driver's alertness. For example, the data processing module 102 can be integrated into a vehicle based computer system (e.g., a car-computer system). The vehicle based computer system can establish communications with a wearable brainwave sensor system (e.g., wearable device 200 of FIG. 2). As discussed above, the data processing module 102 can use accelerometer data from non-brainwave sensors 106 on the wearable device 200 to remove head movement signals from the brainwave data received from the wearable device 200. The data processing module can use the filtered brainwave data to determine when a driver's attentiveness is fading, for example, when the driver is becoming too tired to continue driving safely and present a notification to the driver to pull over and take a break. For example, the vehicle computing system may make an audio recommendation through the vehicle's stereo system or present a message on a navigation display in the vehicle. The data processing module 102 may also receive video data of the user, for example, from camera in the rearview mirror of the vehicle. The data fusion module may use the video data to track the driver's blink rate and use the blink rate data in conjunction with the filtered brainwave data to determine when the driver's level of attentiveness is not sufficient for the driver to continue driving safely.

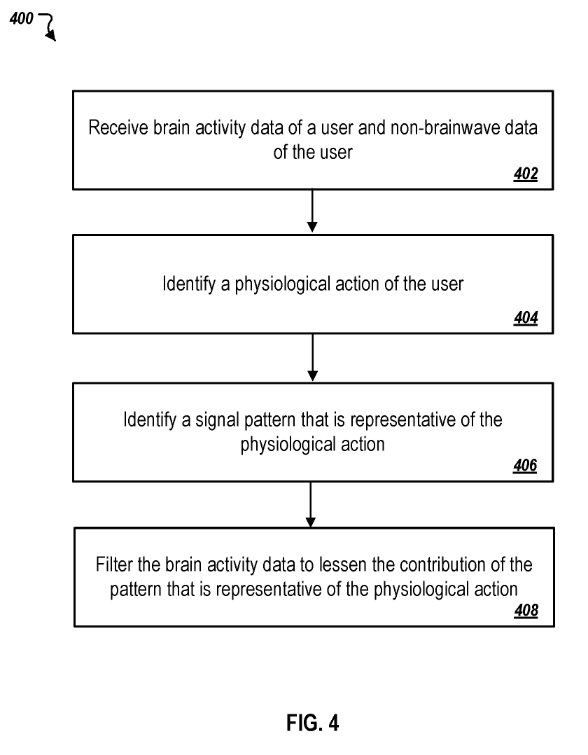

FIG. 4 depicts a flowchart of an example process for filtering brainwave brainwave data. In some implementations, the process 400 can be provided as one or more computer-executable programs executed using one or more computing devices. In some examples, the process 400 is executed by a system such data processing module 102 of FIG. 1, or a computing device such as computing device 118 or wearable device 200 of FIGS. 1 and 2.

The system receives brain activity data of a user from brainwave sensors and user physiological data from non-brainwave sensors (402). The brain activity data represents brainwaves of the user. For example, the brain activity data is an aggregate electrical signal that can represent a signal pattern related to a physiological activity of the user and a brainwave pattern related to a mental activity of the user. The two signal patterns may not be easily recognizable and may interfere with each other. For example, the signal pattern related to the physiological activity of the user may be viewed as noise with respect to the signal pattern related to the mental activity of the user, or vice versa depending on which signal pattern is desired for further analysis. For example, the brainwave data can include brainwaves that are related to the mental activity of a user (e.g., Alpha brainwaves, Gamma brainwaves, Beta brainwaves, Delta brainwaves, and Theta brainwaves). Alpha brainwaves are associated with lapses in attention and sleepiness. Gamma brainwaves are associated with cognitive activity, such as mental calculation. Beta brainwaves may be associated with alertness or anxious thinking. Delta brainwaves are characteristic of slow wave sleep. Theta brainwave phase may be associated with the commission of a cognitive error and theta activity is greater during high levels of alertness to auditory stimulation.

The brainwave data signal can also include interference from noise or other signal patterns related to a user's physiological actions. For example, the data processing module 102 can use data from the non-brainwave sensors 106 to filter noise due to user movements from the brainwave data. For example, user head movements may create noise in the brainwave data. For example, user physiological actions such as muscular movements (e.g., in the face, head, and eyes), heartbeats, and respiration create noise in the brainwave signals received by brainwave sensors 104. The noise may be due to other electrical signals in the body (e.g., nervous system impulses to control muscle movements), other brain signals for controlling such physiological actions, or both.

The system identifies a physiological action of the user (404). For example, the system can identify a physiological action of the user based on the user physiological data from non-brainwave sensors. For example, the system can identify user movements (e.g., head, eye, and/or facial movements), heartbeat, respiration, or a combination thereof. The system can identify the type of user physiological action based on the sensor data. For example, the system can identify that a user moved their head based on data from accelerometers on a wearable device on the user's head. The system can identify that a user moved either eyes based on data from an eye tracking sensor or by processing image data with image processing algorithms (e.g., object detection and tracking algorithms). The system can identify that a user moved their facial muscles based by processing image data (e.g., frames of video) using facial detection algorithms. The system can identify a user's heartbeat based on data from a pulse sensor or by processing images of a user using pulse detection algorithms.

The system identifies a signal pattern that is representative of the physiological action within the brain activity data (406). For example, the system can use a machine learning model to identify signal patterns associated with the identified type of user physiological action. For example, the system can correlate identified user physiological actions with signal patterns within the brainwave data that are related to the identified actions. For example, the system can identify noise patterns generated in brainwave sensors when a user moves their head. As another example, the system can identify interference patterns within the brainwave data caused by a user's heartbeat based on heart rate data such as data indicating a user's pulse rate and timing.

The system filters the brain activity data to lessen a contribution of the pattern that is representative of the identified physiological action to the brain activity data (408). For example, the system can filter the brain activity data to reduce or eliminate the effects of the noise or interference signal pattern caused by the identified user physiological action. For example, the system can use matched filters to reduce the effects of an identified signal pattern, band pass filters to remove spectral effects of the identified signal pattern, other filtering techniques, or a combination thereof to reduce or remove the effects of the identified signal pattern. The system can provide the filtered brain activity data to another computing device. For example, the system can transmit the filtered brain activity data to another computing device. The system can provide the filtered brain activity data to a software application that is executed by the system. In some implementations, the system can use a computer learning model to filter the brain activity data after identifying the signal patterns that represent the user's physiological action.

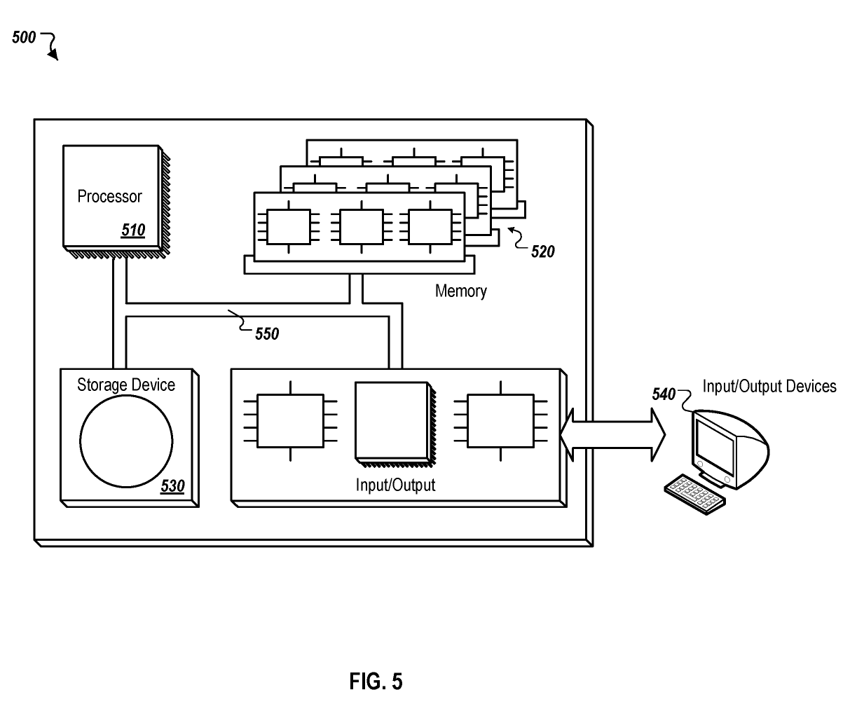

FIG. 5 is a schematic diagram of a computer system 500. The system 500 can be used to carry out the operations described in association with any of the computer-implemented methods described previously, according to some implementations. In some implementations, computing systems and devices and the functional operations described in this specification can be implemented in digital electronic circuitry, in tangibly-embodied computer software or firmware, in computer hardware, including the structures disclosed in this specification (e.g., system 500) and their structural equivalents, or in combinations of one or more of them. The system 500 is intended to include various forms of digital computers, such as laptops, desktops, workstations, personal digital assistants, servers, blade servers, mainframes, and other appropriate computers, including vehicles installed on base units or pod units of modular vehicles. The system 500 can also include mobile devices, such as personal digital assistants, cellular telephones, smartphones, and other similar computing devices. Additionally, the system can include portable storage media, such as, Universal Serial Bus (USB) flash drives. For example, the USB flash drives may store operating systems and other applications. The USB flash drives can include input/output components, such as a wireless transducer or USB connector that may be inserted into a USB port of another computing device.

The system 500 includes a processor 510, a memory 520, a storage device 530, and an input/output device 540. Each of the components 510, 520, 530, and 540 are interconnected using a system bus 550. The processor 510 is capable of processing instructions for execution within the system 500. The processor may be designed using any of a number of architectures. For example, the processor 510 may be a CISC (Complex Instruction Set Computers) processor, a RISC (Reduced Instruction Set Computer) processor, or a MISC (Minimal Instruction Set Computer) processor.

In one implementation, the processor 510 is a single-threaded processor. In another implementation, the processor 510 is a multi-threaded processor. The processor 510 is capable of processing instructions stored in the memory 520 or on the storage device 530 to display graphical information for a user interface on the input/output device 540.

The memory 520 stores information within the system 500. In one implementation, the memory 520 is a computer-readable medium. In one implementation, the memory 520 is a volatile memory unit. In another implementation, the memory 520 is a non-volatile memory unit.

The storage device 530 is capable of providing mass storage for the system 500. In one implementation, the storage device 530 is a computer-readable medium. In various different implementations, the storage device 530 may be a floppy disk device, a hard disk device, an optical disk device, or a tape device.

The input/output device 540 provides input/output operations for the system 500. In one implementation, the input/output device 540 includes a keyboard and/or pointing device. In another implementation, the input/output device 540 includes a display unit for displaying graphical user interfaces.

The features described can be implemented in digital electronic circuitry, or in computer hardware, firmware, software, or in combinations of them. The apparatus can be implemented in a computer program product tangibly embodied in an information carrier, e.g., in a machine-readable storage device for execution by a programmable processor; and method steps can be performed by a programmable processor executing a program of instructions to perform functions of the described implementations by operating on input data and generating output. The described features can be implemented advantageously in one or more computer programs that are executable on a programmable system including at least one programmable processor coupled to receive data and instructions from, and to transmit data and instructions to, a data storage system, at least one input device, and at least one output device. A computer program is a set of instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

Suitable processors for the execution of a program of instructions include, by way of example, both general and special purpose microprocessors, and the sole processor or one of multiple processors of any kind of computer. Generally, a processor will receive instructions and data from a read-only memory or a random access memory or both. The essential elements of a computer are a processor for executing instructions and one or more memories for storing instructions and data. Generally, a computer will also include, or be operatively coupled to communicate with, one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including by way of example semiconductor memory devices, such as EPROM, EEPROM, and flash memory devices; magnetic disks such as internal hard disks and removable disks; magneto-optical disks; and CD-ROM and DVD-ROM disks. The processor and the memory can be supplemented by, or incorporated in, ASICs (application-specific integrated circuits).

To provide for interaction with a user, the features can be implemented on a computer having a display device such as a CRT (cathode ray tube) or LCD (liquid crystal display) monitor for displaying information to the user and a keyboard and a pointing device such as a mouse or a trackball by which the user can provide input to the computer. Additionally, such activities can be implemented via touchscreen flat-panel displays and other appropriate mechanisms.

The features can be implemented in a computer system that includes a back-end component, such as a data server, or that includes a middleware component, such as an application server or an Internet server, or that includes a front-end component, such as a client computer having a graphical user interface or an Internet browser, or any combination of them. The components of the system can be connected by any form or medium of digital data communication such as a communication network. Examples of communication networks include a local area network (“LAN”), a wide area network (“WAN”), peer-to-peer networks (having ad-hoc or static members), grid computing infrastructures, and the Internet.

The computer system can include clients and servers. A client and server are generally remote from each other and typically interact through a network, such as the described one. The relationship of client and server arises by virtue of computer programs running on the respective computers and having a client-server relationship to each other.

While this specification contains many specific implementation details, these should not be construed as limitations on the scope of any inventions or of what may be claimed, but rather as descriptions of features specific to particular implementations of particular inventions. Certain features that are described in this specification in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

Thus, particular implementations of the subject matter have been described. Other implementations are within the scope of the following claims. In some cases, the actions recited in the claims can be performed in a different order and still achieve desirable results. In addition, the processes depicted in the accompanying figures do not necessarily require the particular order shown, or sequential order, to achieve desirable results. In certain implementations, multitasking and parallel processing may be advantageous.

What is claimed is:

1. A computer-implemented brainwave filtering method executed by one or more processors and comprising:
receiving brain activity data of a user from a brainwave sensor and user physiological data from a non-brainwave sensor, the brain activity data representing a brainwave pattern related to a physiological activity of the user and a brainwave pattern related to a mental activity of the user;
identifying, based on the user physiological data, a physiological action of the user;
identifying, within the brain activity data, a pattern that is representative of the identified physiological action; and
filtering the brain activity data to lessen a contribution of the pattern representative of the identified physiological action to the brain activity data, thereby, providing filtered brain activity data.

2. The method of claim 1, wherein the non-brainwave sensor includes a sensor selected from the group consisting of: a motion sensor, an accelerometer, a camera, a radar sensor, a microphone, a blood pressure sensor, a pulse sensor, and a skin conductance sensor.

3. The method of claim 1, wherein physiological action includes an action selected from the group consisting of: a head movement, a movement of facial muscles, a pulse rate, and an eye movement.

4. The method of claim 1, wherein identifying the pattern that is representative of the identified physiological action and filtering the brain activity data are performed by a machine learning system.

5. The method of claim 4, wherein the machine learning system is a feed forward auto encoder neural network.

6. The method of claim 1, further comprising identifying, based on correlation between the identified physical action and the filtered brain activity data, a brain state of the user.

7. The method of claim 6, wherein the identified physiological action is an action selected from the group consisting of: eye movement, a blink rate, perspiration, and a keyboard typing intensity, and wherein the brain state is a level of user attentiveness.

8. The method of claim 1, further comprising prompting the user to perform an action based on determining the brain state of the user.

9. The method of claim 1, wherein the brainwave sensor is part of a brainwave sensor system and the brain activity data is received from the brainwave sensor system.

10. The method of claim 9, wherein the brainwave sensor system is a wearable brainwave sensor system comprising a plurality of electrodes arranged in a comb-like structure.

11. The method of claim 10, wherein the electrodes are retractable.

12. The method of claim 10, wherein the non-brainwave sensor is a motion sensor mounted on the brainwave sensor system.

13. A system comprising:
a brainwave sensor;
at least one non-brainwave sensor; and
a data processing module communicably coupled to the brainwave sensor and the at least one non-brainwave sensor, the data processing module comprising:
a physiological action detection module configured to identify a physiological action of the user based on user physiological data received from the at least one non-brainwave sensor; and
a filtering module configured to:
identify, within brain activity data received from the brainwave sensor, a pattern representative of the physiological action of the user, and
filter the brain activity data to lessen a contribution of the pattern representative of the identified physiological action to the brain activity data to provide filtered brain activity data.

14. The system of claim 13 wherein the data processing module comprises a data fusion module configured to identify a brain state of the user based on a correlation between the physiological data and the filtered brain activity data.

15. The system of claim 14 wherein the data processing module comprises an output module configured to present, to a user, a prompt to perform an action based on the determined brain state of the user.

16. The system of claim 13, wherein the non-brainwave sensors include a sensor selected from the group consisting of: a motion sensor, an accelerometer, a camera, a radar sensor, a microphone, a blood pressure sensor, a pulse sensor, and a skin conductance sensor.

17. The system of claim 13, wherein the physiological action includes an action selected from the group consisting of: a head movement, a movement of facial muscles, a pulse rate, and an eye movement.

18. The system of claim 13, wherein the filtering module comprises a machine learning system.

19. The system of claim 18, wherein the machine learning system is configured to identify the pattern that is representative of the identified physiological action and filter the brain activity data.

20. A system comprising:
a brainwave sensor;
at least one non-brainwave sensor;
a data processing module communicably coupled to the brainwave sensor system and the at least one non-brainwave sensor; and
a data store coupled to the data processing module having instructions stored thereon which, when executed by the data processing module, causes the data processing module to perform operations comprising:
receiving brain activity data of a user from the brainwave sensor and user physiological data from the at least one non-brainwave sensor, the brain activity data representing a brainwave pattern related to a physiological activity of the user and a brainwave pattern related to a mental activity of the user;
identifying, based on the user physiological data, a physical action of the user;
identifying, within the brain activity data, a pattern that is representative of the identified physical action; and
filtering the brain activity data to lessen a contribution of the pattern representative of the identified physiological action to the brain activity data.
